(19) United States
(12) Patent Application Publication
(10) Pub. No.: US 2018/0161005 A1
(43) Pub. Date: Jun. 14, 2018

(54) ULTRASONIC PROBE

(71) Applicant: Seiko Epson Corporation, Tokyo (JP)

(72) Inventor: Masayoshi YAMADA, Chino (JP)

(21) Appl. No.: 15/839,228

(22) Filed: Dec. 12, 2017

(30) Foreign Application Priority Data
Dec. 14, 2016 (JP) 2016-241960

(51) Int. Cl. A61B 8/00; A61B 8/08; B06B 1/06; H01L 41/08; H01L 41/332; G10K 11/02

(52) U.S. Cl. CPC A61B 8/4427; A61B 8/4494; A61B 8/4488; A61B 8/52; G10K 11/02; A61B 8/461; H01L 41/081; H01L 41/332; B06B 1/0629

(57) ABSTRACT

An ultrasonic probe includes an acoustic lens, which is positioned in an emission direction of the ultrasonic waves emitted by an ultrasonic element and has end face in a direction intersecting the emission direction, and a resin portion which is opposed to acoustic lens end face and which includes a filler and resin in a case where the end face of the acoustic lens is set as the acoustic lens end face.

### BACKGROUND

### 1. Technical Field

The present invention relates to an ultrasonic probe.

### 2. Related Art

In the related art, for example, an ultrasonic device including an ultrasonic element as disclosed in JP-A-7-178083 is generally known. The ultrasonic device includes a first substrate and the first substrate has an element array that includes ultrasonic elements arranged in an array. The element array is covered with an acoustic matching layer. The acoustic matching layer couples an acoustic lens to the element array. A second substrate is installed on a surface located at a side opposite to a surface on which the element array is installed in the first substrate and the second substrate reinforces the stiffness of the first substrate. As such, the second substrate, the first substrate, the acoustic matching layer, and the acoustic lens are installed to be superimposed on one another in the ultrasonic device.

The ultrasonic probe is configured with the ultrasonic device, a driving circuit, a probe casing, and the like. The driving circuit is a circuit to drive the element array. The ultrasonic device and the driving circuit are installed in the inside of the probe casing and the acoustic lens is disposed to be exposed from the ultrasonic probe.

A groove portion between the ultrasonic device and the probe casing is filled with silicone rubber in the ultrasonic probe described in JP-A-7-178083. An effect of attenuating the ultrasonic waves by silicone rubber simple substance is small. The ultrasonic waves emitted by the ultrasonic element includes the unnecessary ultrasonic waves advancing toward an acoustic lens end face as well as the ultrasonic waves advancing in an emission direction. The unnecessary ultrasonic waves are reflected on the acoustic lens end face. The unnecessary ultrasonic waves reflected on the acoustic lens end face reach an ultrasonic element by being delayed for a predetermined time from the time when emission is performed. For that reason, accuracy of ultrasonic wave measurement is lowered. The ultrasonic probe in which reflection of the ultrasonic waves on the acoustic lens end face is suppressed and accuracy of ultrasonic wave measurement can be improved was expected.

### SUMMARY

An advantage of some aspects of the invention is to solve at least a part of the problems described above, and the invention can be implemented as the following forms or application examples.

### APPLICATION EXAMPLE 1

An ultrasonic probe according to this application example includes an acoustic lens which is positioned in an emission direction of ultrasonic waves emitted by an ultrasonic element and has end face in a direction intersecting the emission direction, and a resin portion which is opposed to acoustic lens end face which is the end face of the acoustic lens and includes a filler and resin.

According to this application example, the ultrasonic probe includes an ultrasonic device, a probe casing, and the resin portion. The ultrasonic device includes an acoustic lens, an acoustic matching layer, a first substrate, and a second substrate. The ultrasonic element is vibrated by ultrasonic waves at the time of emitting the ultrasonic waves. The ultrasonic waves are transferred from the ultrasonic element to the acoustic lens and are emitted from the surface of the ultrasonic probe. The acoustic lens is positioned in the emission direction of the ultrasonic waves emitted by the ultrasonic element. The acoustic lens plays a role of converging the ultrasonic waves emitted from the ultrasonic device. The unnecessary ultrasonic waves propagate also in a direction intersecting the emission direction of the ultrasonic waves emitted by the ultrasonic element, in the inside of the acoustic lens.

The acoustic lens has the acoustic lens end face in the direction intersecting the emission direction of the ultrasonic waves. The ultrasonic probe includes a resin portion which is opposed to acoustic lens end face and includes a filler and resin. The resin portion is positioned on a groove portion between the ultrasonic device and the probe casing. The filler is included in the resin portion and the ultrasonic waves are irregularly reflected within the resin portion by the filler. The ultrasonic waves are attenuated while advancing in the inside of the resin portion. By doing this, the resin portion absorbs the ultrasonic waves. For that reason, the ultrasonic waves advancing toward an acoustic lens end face is absorbed by the resin portion. Reflection of the ultrasonic waves on the acoustic lens end face is effectively attenuated. The resin portion containing the filler is installed on the acoustic lens end face and thus, reflection of the ultrasonic waves is suppressed. As a result, reflection of the ultrasonic waves on the acoustic lens end face is suppressed and thus, accuracy of ultrasonic wave measurement may be improved.

### APPLICATION EXAMPLE 2

In the ultrasonic probe according to the application example, it is preferable that the acoustic lens includes a filler and in a case where the filler included in the acoustic lens is set as a first filler and the filler included in the resin portion is set as a second filler, the number-average diameter of the second filler is larger than the number-average diameter of the first filler.

According to this application example, the filler is included in the acoustic lens and the resin portion. Also, the number-average diameter is an average value of the two-values-average particle diameter of each filter. When a length of a long axis of the filler is set as I and a length of a short axis thereof is set as B, the two-values-average particle diameter is expressed by two-values-average particle diameter=(I+B)/2. As the number-average diameter of the fillers becomes large, the ultrasonic waves are gradually scattered to be significantly attenuated. In a case where the filler included in the acoustic lens is set as the first filler and the filler included in the resin portion is set as the second filler, a number-average diameter relationship of the filler for the second filler is larger than that of the first filler and thus, the ultrasonic waves are irregularly reflected more easily by using the resin portion rather than the acoustic lens. Accordingly, it is possible to effectively attenuate the ultrasonic waves by using the resin portion rather than the acoustic lens. As a result, it is possible to suppress reflection of the ultrasonic waves on the acoustic lens end face.

### APPLICATION EXAMPLE 3

In the ultrasonic probe according to the application example described above, it is preferable that the material of the resin portion includes at least any one of silicone rubber, foamed silicone, and epoxy resin and the filler included in the resin portion includes at least any one of powders of the group consisting of zinc oxide powder, zirconium oxide powder, alumina powder, silica powder, titanium oxide powder, silicon carbide powder, aluminum nitride powder, carbon powder, calcium carbonate powder, and boron nitride powder.

According to this application example, at least any one of silicone rubber, foamed silicone, and epoxy resin is included as the material of the resin portion. Silicone rubber, foamed silicone, and epoxy resin have a nature of maintaining a state of being viscous body before curing and becoming solid after curing and thus, the filler may be easily mixed before curing. Furthermore, moisture or foreign substances may be prevented from entering the inside of the ultrasonic probe. Accordingly, the resin portion has a nature of maintaining a state of being viscous body before curing and becoming solid after curing and thus, the filler may be easily mixed in the resin portion before curing. Furthermore, moisture or foreign substances may be prevented from entering the inside of the ultrasonic probe.

At least any one of powders of a group of zinc oxide powder, zirconium oxide powder, alumina powder, silica powder, titanium oxide powder, silicon carbide powder, aluminum nitride powder, carbon powder, calcium carbonate powder, and boron nitride powder is included in the filler, which is included in the resin portion. Various fillers described above may scatter and attenuate the ultrasonic waves in the inside of the resin portion. Accordingly, the resin portion containing the fillers may attenuate the ultrasonic waves.

### APPLICATION EXAMPLE 4

In the ultrasonic probe according to the application example described above, it is preferable that a first substrate on which the ultrasonic element is installed and a second substrate for improving the stiffness of the first substrate are installed in this order at a side opposite to the emission direction of the ultrasonic waves of the acoustic lens, the first substrate and the second substrate have end faces in a direction intersecting the emission direction, and in a case where the end face of the first substrate is set as a first substrate end face and the end face of the second substrate is set as a second substrate end face, the resin portion is opposed to the first substrate end face and the second substrate end face.

According to this application example, the ultrasonic probe includes the first substrate and the second substrate at an opposite side of the emission direction of the ultrasonic waves. The ultrasonic element is installed on the first substrate. The second substrate plays a role of improving the stiffness of the first substrate. The ultrasonic waves emitted from the ultrasonic element propagate also inside of the second substrate and are reflected also on the second substrate end face. The ultrasonic waves emitted from the ultrasonic element are reflected also on the first substrate end face of the first substrate on which the ultrasonic element is installed. The resin portion is extended to the place opposing the first substrate end face and the second substrate end face so as to make it possible to effectively attenuate reflection of the ultrasonic waves on the second substrate end face or the first substrate end face of the first substrate on which the ultrasonic element is installed.

### APPLICATION EXAMPLE 5

An ultrasonic probe according to this application example includes an acoustic lens which is positioned in an emission direction of the ultrasonic waves emitted by an ultrasonic element and has an acoustic lens end face in the direction intersecting the emission direction and a recessed and projecting member which is opposed to the acoustic lens end face and has unevenness.

According to this application example, the ultrasonic probe includes the ultrasonic elements and the acoustic lens. The ultrasonic element is vibrated by ultrasonic waves at the time of emitting the ultrasonic waves. The ultrasonic waves are transferred from the ultrasonic element to the acoustic lens and are emitted from the surface of the ultrasonic probe. The acoustic lens is positioned in the emission direction of the ultrasonic waves emitted by the ultrasonic element. The acoustic lens plays a role of converging the ultrasonic waves emitted from the ultrasonic device. The unnecessary ultrasonic waves propagate also in a direction intersecting the emission direction of the ultrasonic waves emitted by the ultrasonic element in the inside of the acoustic lens.

The acoustic lens has the acoustic lens end face in the direction intersecting the emission direction of the ultrasonic waves. The acoustic lens end face is the end face of the acoustic lens. The recessed and projecting member is opposed to the acoustic lens end face. The recessed and projecting member irregularly reflects the ultrasonic waves. For that reason, the ultrasonic waves advancing toward an acoustic lens end face are irregularly reflected and attenuated by the recessed and projecting member. An amount of the ultrasonic waves advancing toward the acoustic lens end face and reflected on the acoustic lens end face is effectively attenuated. Accordingly, the recessed and projecting member is installed on the end face of the probe casing and thus, reflection of the ultrasonic waves is suppressed. As a result, reflection of the ultrasonic waves on the acoustic lens end face is suppressed and thus, accuracy of ultrasonic wave measurement may be improved.

### APPLICATION EXAMPLE 6

In the ultrasonic probe according to the application example described above, it is preferable that a cross-section of unevenness of the recessed and projecting member is a triangle in at least one of a direction opposed and orthogonal to or parallel with the acoustic lens end face.

According to this application example, the ultrasonic wave includes the acoustic lens and the recessed and projecting member. When a cross-section in at least one of the orthogonal direction and the parallel direction with respect to the acoustic lens end face is a triangle, the recessed and projecting member can irregularly reflect the ultrasonic waves effectively. Accordingly, the ultrasonic waves advancing toward the acoustic lens end face is irregularly reflected on the recessed and projecting member and thus, reflection of the ultrasonic waves on the acoustic lens end face is effectively attenuated.

### APPLICATION EXAMPLE 7

In the ultrasonic probe according to the application example described above, it is preferable that a first substrate on which the ultrasonic element is installed and a second substrate for improving the stiffness of the first substrate are installed in this order at a side opposite to the emission direction of the ultrasonic waves of the acoustic lens, the first substrate and the second substrate have end faces in a direction intersecting the emission direction, and in a case where the end face of the first substrate is set as the first substrate end face and the end face of the second substrate is set as the second substrate end face, the recessed and projecting member is opposed to the first substrate end face and the second substrate end face.

According to this application example, the ultrasonic probe includes the first substrate and the second substrate at the opposite side of the emission direction of the ultrasonic waves. The ultrasonic element is installed on the first substrate. The second substrate plays a role of improving the stiffness of the first substrate. The ultrasonic waves emitted from the ultrasonic element propagate also inside of the second substrate and are reflected also on the second substrate end face. The ultrasonic waves emitted from the ultrasonic element are reflected also on the first substrate end face of the first substrate on which the ultrasonic element is installed. The recessed and projecting member is extended to the place opposing the first substrate and the second substrate so as to make it possible to effectively attenuate reflection of the ultrasonic waves on the second substrate end face or the first substrate end face.

### APPLICATION EXAMPLE 8

In the ultrasonic probe according to the application example described above, it is preferable that a probe casing is installed to be opposed to the acoustic lens end face and the recessed and projecting member is integrated with the probe casing.

According to this application example, the probe casing is preferably installed to be opposed to the acoustic lens end face, in the ultrasonic probe. The recessed and projecting member is included to be opposed to the acoustic lens end face, in the ultrasonic probe. The recessed and projecting member irregularly reflects the ultrasonic waves. For that reason, the ultrasonic waves advancing toward the acoustic lens end face are irregularly reflected on the recessed and projecting member and thus, reflection of the ultrasonic waves on the acoustic lens end face can be effectively attenuated. Since the recessed and projecting member is integrated with the end face of the probe casing, the number of components can be reduced compared to a case where the recessed and projecting member and the end face of the probe casing are formed at different portions and accordingly, it is possible to manufacture the ultrasonic probe with high productivity.

### BRIEF DESCRIPTION OF THE DRAWINGS

The invention will be described with reference to the accompanying drawings, wherein like numbers reference like elements.

FIG. 1 is a perspective view schematically illustrating a configuration of an ultrasonic diagnosis apparatus according to Embodiment 1.

FIG. 2 is a side view schematically illustrating a configuration of an ultrasonic probe.

FIG. 3 is a cross-sectional view schematically illustrating a structure of a probe head.

FIG. 4 is a plan view schematically illustrating the ultrasonic probe.

FIG. 5 is a plan view schematically illustrating a configuration of a first substrate.

FIG. 6 is a cross-sectional view schematically illustrating a function of a resin portion.

FIG. 7 is a plan view schematically illustrating an ultrasonic probe according to Embodiment 2.

FIG. 8 is a cross-sectional view schematically illustrating a function of a recessed and projecting member.

FIG. 9 is a cross-sectional view schematically illustrating an ultrasonic probe according to Modification Example 3.

FIG. 10 is a cross-sectional view schematically illustrating an ultrasonic probe according to Modification Example 5.

### DESCRIPTION OF EXEMPLARY EMBODIMENTS

In the following, preferred embodiments of the invention will be described in detail using the drawings. In the following respective drawings, in order to represent respective layers and respective members in a size to the extent that respective layers and members can be recognized, scale of respective layers and members and an actual scale thereof are made different.

### Embodiment 1

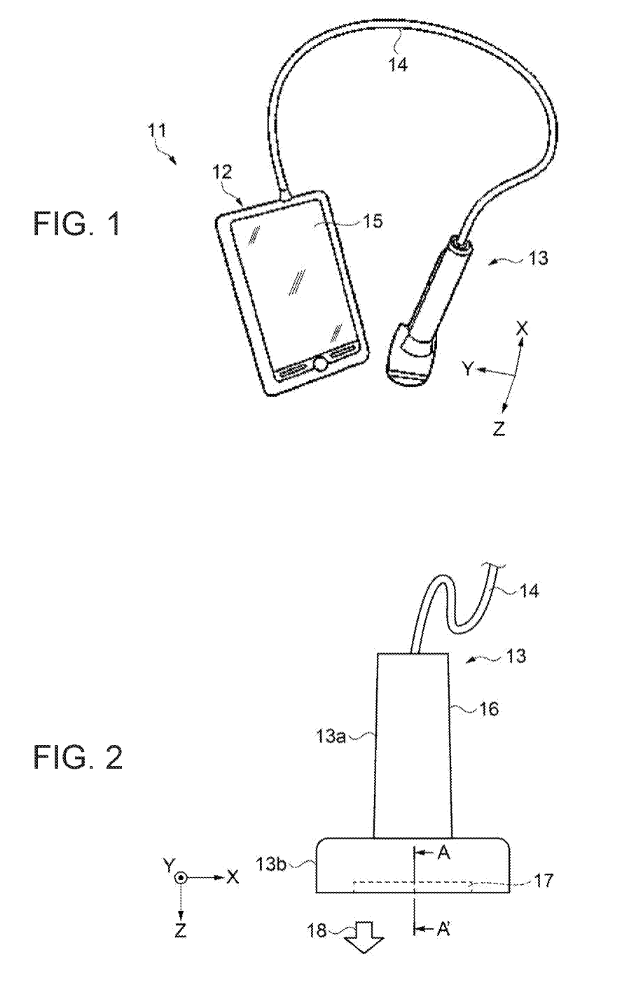

FIG. 1 is a perspective view schematically illustrating a configuration of an ultrasonic diagnosis apparatus according to Embodiment 1 and schematically illustrates a configuration of a specific example of an electronic apparatus according to Embodiment 1, that is, an ultrasonic diagnosis apparatus 11. As illustrated in FIG. 1, the ultrasonic diagnosis apparatus 11 includes an apparatus terminal 12 and an ultrasonic probe 13. The apparatus terminal 12 and the ultrasonic probe 13 are connected with each other by a cable 14. The apparatus terminal 12 and the ultrasonic probe 13 exchange an electrical signal with each other through the cable 14. A display panel 15 is incorporated in the apparatus terminal 12. A screen of the display panel 15 is exposed on the surface of the apparatus terminal 12. An image based on the ultrasonic wave detected by the ultrasonic probe 13 is generated in the apparatus terminal 12. An imaged detection result is displayed on the screen of the display panel 15.

FIG. 2 is a side view schematically illustrating a configuration of the ultrasonic probe 13. As illustrated in FIG. 2, the ultrasonic probe 13 includes a probe casing 16. An ultrasonic device 17 is accommodated in a probe casing 16. The surface of the ultrasonic device 17 is exposed on the surface of the probe casing 16. The ultrasonic device 17 emits an ultrasonic wave from the surface thereof toward an emission direction 18 and receives a reflected wave of the ultrasonic waves. Also, the ultrasonic probe 13 includes a probe head 13b detachably connected to a probe body 13a. In this case, the ultrasonic device 17 is incorporated in the probe casing 16 of the probe head 13b.

A plane shape of the ultrasonic device 17 is a rectangular plate shape. A longitudinal direction in a planar direction of the ultrasonic device 17 is set as the X-direction and a direction orthogonal to the X-direction is set as the Y-direction. A thickness direction of the ultrasonic device 17 is set as the Z-direction. The Z-direction and the emission direction 18 are the same directions.

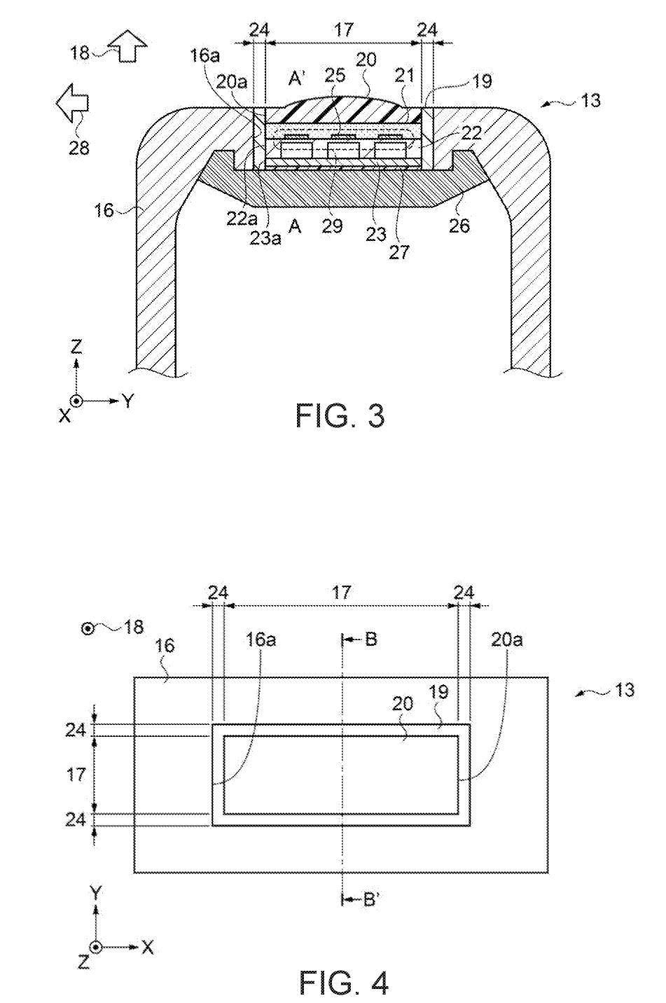

FIG. 3 is a cross-sectional view schematically illustrating a structure of the probe head according to Embodiment 1 and is a cross-sectional view taken along the A-A′ line illustrated in FIG. 2. The ultrasonic probe 13 includes the ultrasonic device 17, the probe casing 16, a support member 26, and a resin portion 19. An outer shape of the probe casing 16 is a long shape in the X-direction when seen from the Z-direction, similar to the ultrasonic device 17. An opening 16a is installed at the central portion of the probe casing 16. The probe casing 16 may be composed of ABS resin, acrylic resin, or a resin material. The probe casing 16 may also be composed of a composite material of silicone rubber and the resin material.

At the opening 16a, the ultrasonic device 17 is installed on the support member 26. A groove portion 24 between the ultrasonic device 17 and the probe casing 16 is filled with the resin portion 19. The ultrasonic device 17 and the support member 26 are fixed by an adhesive member 27. For example, ABS resin or acrylic resin is used for the support member 26. An epoxy-based adhesive or a double-sided adhesive tape is used for the adhesive member 27. The ultrasonic device 17 includes a first substrate 22 having an element array 29 that includes ultrasonic elements 25 arranged in an array. The ultrasonic element 25 emits the ultrasonic waves in the emission direction 18. The element array 29 is covered with an acoustic matching layer 21. The acoustic matching layer 21 couples the acoustic lens 20 to the element array 29. The acoustic lens 20 is positioned in the emission direction 18 of the ultrasonic waves emitted by the ultrasonic element 25. For example, silicone rubber can be used for the acoustic matching layer 21 and the acoustic lens 20. A value of acoustic impedance of the acoustic lens 20 is close to the value of acoustic impedance of the resin portion 19 or a living body.

The first substrate 22 on which the ultrasonic element 25 is installed and a second substrate 23 for improving the stiffness of the first substrate 22 are installed in this order at a side opposite to the emission direction 18 of the ultrasonic waves of the acoustic lens 20. The first substrate 22 and the second substrate 23 have end faces in a direction 28 intersecting the emission direction 18. The end face of the first substrate 22 is set as a first substrate end face 22a and the end face of the second substrate 23 is set as a second substrate end face 23a. The resin portion 19 is opposed to the first substrate end face 22a and the second substrate end face 23a. As such, the second substrate 23, the first substrate 22, the acoustic matching layer 21, and the acoustic lens 20 are installed to be superimposed on one another and the ultrasonic device 17 is configured with these components.

The acoustic lens 20 has acoustic lens end face 20a in a direction 28 intersecting the emission direction 18. The acoustic lens end face 20a is the end face positioned in the X-direction and the Y-direction of the acoustic lens 20. The ultrasonic probe 13 includes the resin portion 19 which is opposed to the acoustic lens end face 20a and includes a filler and resin.

As the material of the resin portion 19, at least any one of silicone rubber, foamed silicone, and epoxy resin is included. Silicone rubber, foamed silicone, and epoxy resin have a nature of maintaining a state of being viscous body before curing and becoming solid after curing and thus, the filler can be easily mixed before curing. Furthermore, moisture or foreign substances can be prevented from entering the inside of the ultrasonic probe 13. Accordingly, the filler can be easily mixed in the resin portion 19 before curing and furthermore, moisture or foreign substances can be prevented from entering the inside of the ultrasonic probe 13, by the resin portion 19.

At least any one of powders of a group of zinc oxide powder, zirconium oxide powder, alumina powder, silica powder, titanium oxide powder, silicon carbide powder, aluminum nitride powder, carbon powder, calcium carbonate powder, and boron nitride powder is included in the filler, which is included in the resin portion 19. Various fillers described above can scatter and attenuate the ultrasonic waves in the inside of the resin portion 19. For that reason, the resin portion 19 can attenuate the ultrasonic waves.

FIG. 4 is a plan view schematically illustrating the ultrasonic probe according to Embodiment 1. The ultrasonic device 17 is installed at the opening 16a of the probe casing 16. When the ultrasonic device 17 is installed at the opening 16a, a groove portion 24 is formed between the probe casing 16 and the acoustic lens 20. The resin portion 19 filled with resin is formed in the groove portion 24. The acoustic lens 20 installed in the ultrasonic device 17 is disposed to be exposed from the ultrasonic probe 13.

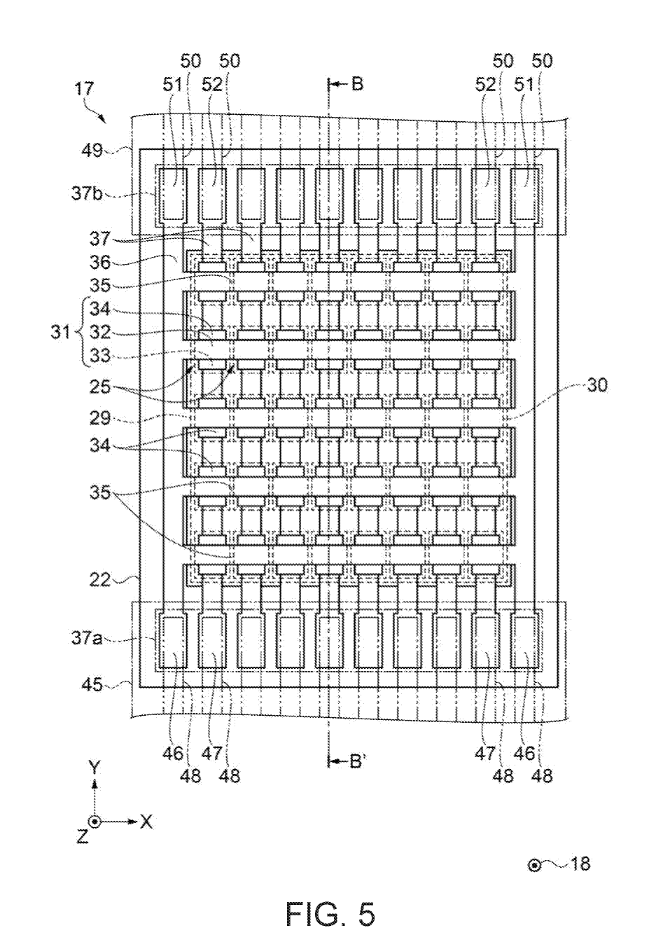

FIG. 5 is a plan view schematically illustrating a configuration of a first substrate. The ultrasonic device 17 includes a first substrate 22. An element array 29 is formed in the first substrate 22. The element array 29 is configured with an array of the ultrasonic elements 25 arranged in an array. The array is formed in a matrix composed of a plurality of rows and a plurality of columns. Also, a staggered-pattern arrangement is established in the array. In the staggered-pattern arrangement, a group of the ultrasonic elements 25 in even-numbered rows may be shifted with respect to a group of the ultrasonic elements 25 in odd-numbered rows by one-half of a row pitch.

Each ultrasonic element 25 includes a diaphragm 30. In the FIG. 5, a contour of the diaphragm 30 is plotted in a dotted line in plan view (plan view from the thickness direction of the first substrate) in a direction orthogonal to a film surface of the diaphragm 30. A piezoelectric element 31 is formed on the diaphragm 30. The piezoelectric element 31 is configured with an upper electrode 32, a lower electrode 33, and a piezoelectric film 34. The piezoelectric film 34 is sandwiched between the upper electrode 32 and the lower electrode 33 for each ultrasonic element 25. These components are superposed in order of the lower electrode 33, the piezoelectric film 34, and the upper electrode 32.

A plurality of first conductors 35 are formed on the surface of the first substrate 22. The first conductors 35 extend in parallel with each other in the direction of row of the arrangement. One first conductor 35 is allocated for every ultrasonic element 25 of one row. One first conductor 35 is commonly connected to the piezoelectric films 34 of the ultrasonic elements 25 aligned in the row direction. The first conductor 35 allows the upper electrode 32 to be formed on each ultrasonic element 25. Both ends of the first conductor 35 are respectively connected to a pair of lead-out wirings 36. Lead-out wirings 36 extend in parallel with each other in the direction of column of the arrangement. Accordingly, all first conductors 35 have the same length. As such, the upper electrode 32 is commonly connected to the ultrasonic elements 25 of the entire matrix. The first conductor 35 can be made of, for example, iridium (Ir). However, other conductive materials may be used for the first conductor 35. The first conductor 35 and the lead-out wiring 36 are formed by patterning a laminated conductive layer. The material of the first substrate 22 can be composed of, for example, silicone (Si). As the material of the second substrate 23, for example, silicone (Si), 42 alloy, a stainless metal plate can be used. The piezoelectric film 34 is made of, for example, lead zirconate titanate (PZT).

A plurality of second conductors 37 are formed on the surface of the first substrate 22. The second conductors 37 extend in parallel with each other in the direction of column of the arrangement. One second conductor 37 is allocated for every ultrasonic element 25 of one column.

One second conductor 37 is commonly disposed to the piezoelectric films 34 of the ultrasonic elements 25 aligned in the column direction. The second conductor 37 allows the lower electrode 33 to be formed on each ultrasonic element 25. For the second conductor 37, for example, a laminated film of titanium (Ti), iridium (Ir), platinum (Pt), and titanium can be used. However, other conductive materials may be used for the second conductor 37. The second conductor 37 is formed by patterning the laminated conductive layer.

The first substrate 22 is connected with a first wiring board 45 which is a flexible printed wiring board. The first wiring board 45 covers the first terminal array 37a. Conductive lines, that is, first signal lines 48 are formed on one end of the first wiring board 45 by being individually corresponded to the upper electrode terminals 46 and the lower electrode terminal 47. The first signal lines 48 are individually opposed to the upper electrode terminals 46 and the lower electrode terminals 47 and individually joined therewith. Similarly, a second wiring board 49 which is a flexible printed wiring board is covered by the first substrate 22. The second wiring board 49 covers a second terminal array 37b. Conductive lines, that is, second signal lines 50 are formed on one end of the second wiring board 49 by being individually corresponded to the upper electrode terminals 51 and the lower electrode terminal 52. The second signal lines 50 are individually opposed to the upper electrode terminals 51 and the lower electrode terminals 52 and individually joined therewith.

The acoustic matching layer 21 is laminated on the surface of the first substrate 22. A film thickness of the acoustic matching layer 21 is determined by a resonance frequency of the diaphragm 30. The resonance frequency of the diaphragm 30 is determined by a width of the diaphragm 30.

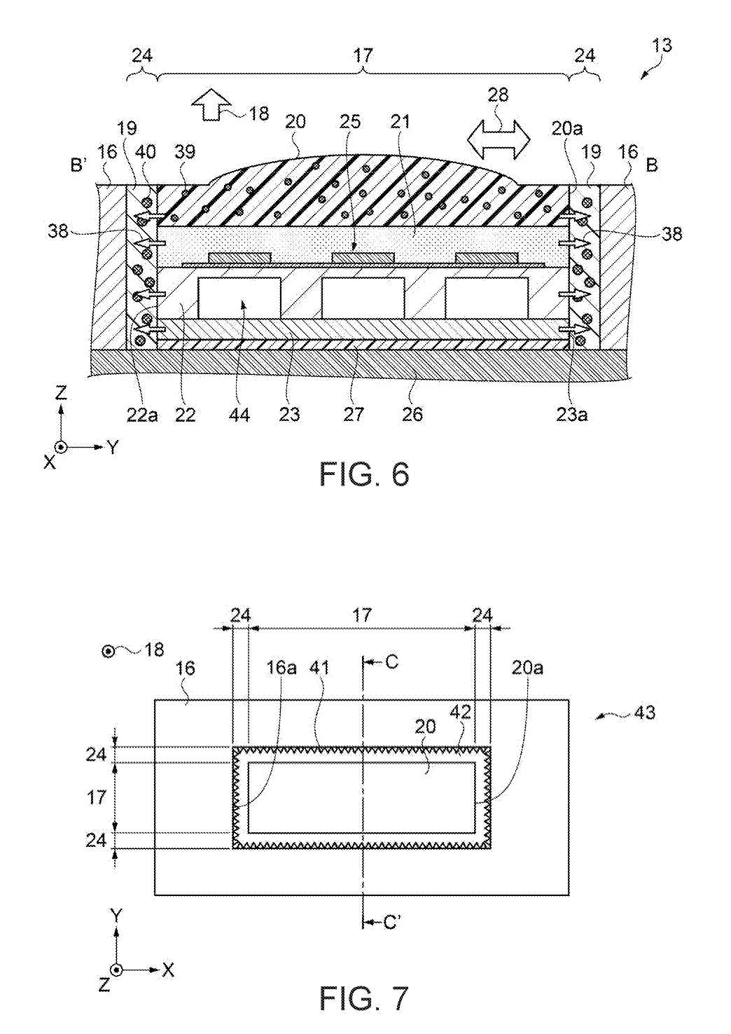

FIG. 6 is a cross-sectional view of the ultrasonic probe 13 schematically illustrating a function of the resin portion and is an enlarged view of the cross-sectional view schematically illustrated in FIG. 3. The second substrate 23, the first substrate 22, the acoustic matching layer 21, and the acoustic lens 20 are installed to be superimposed on one another and the ultrasonic device 17 is configured with these components. The ultrasonic device 17 includes the first substrate 22. Recessed portions 44 are formed in the first substrate 22 in a matrix shape. The first substrate 22 includes the ultrasonic elements 25 at places thereof opposing the recessed portions 44. Although the first substrate 22 is connected with the first wiring board and the second wiring board, the first wiring board and the second wiring boards are omitted in FIG. 6 in order to make it easy to understand the effect of the invention. The ultrasonic element 25 is vibrated by ultrasonic waves at the time of emitting the ultrasonic waves. The ultrasonic waves are transferred from the ultrasonic element 25 to the acoustic lens 20 and are emitted from the surface of the ultrasonic probe 13. The acoustic lens 20 is positioned in the emission direction 18 of the ultrasonic waves emitted by the ultrasonic element 25. The acoustic lens 20 plays a role of converging the ultrasonic waves emitted from the ultrasonic device 17. The unnecessary ultrasonic waves propagate also in a direction 28 intersecting the emission direction of the ultrasonic waves emitted by the ultrasonic element 25 in the inside of the acoustic lens 20.

The groove portion 24 between the probe casing 16 and the ultrasonic device 17 is filled with resin and forms the resin portion 19. The filler is included in the resin portion 19 and the ultrasonic waves are irregularly reflected within the resin portion by the filler. The ultrasonic waves are attenuated while advancing in the inside of the resin portion. By doing this, the resin portion 19 absorbs the ultrasonic waves. For that reason, the ultrasonic waves 38 advancing toward an acoustic lens end face 20a are absorbed by the resin portion 19. Accordingly, reflection of the ultrasonic waves on the acoustic lens end face 20a is effectively attenuated. The resin portion 19 containing the filler is installed on the acoustic lens end face 20a and thus, reflection of the ultrasonic waves is suppressed. As a result, reflection of the ultrasonic waves on the acoustic lens end face 20a is suppressed and thus, accuracy of ultrasonic wave measurement can be improved.

The acoustic lens 20 includes the filler and in a case where the filler included in the acoustic lens 20 is set as a first filler 39 and the filler included in the resin portion 19 is set as a second filler 40, the number-average diameter of the second filler 40 is preferably larger than the number-average diameter of the first filler 39. The number-average diameter is an average value of the two-values-average particle diameter of each filter. When a length of a long axis of the filler is set as I and a length of a short axis thereof is set as, B, the two-values-average particle diameter can be obtained by two-values-average particle diameter=(I +B)/2. The two-values-average particle diameter can be calculated using a microscopic method. The number-average diameter is an average value of the two-values-average particle diameters of each filter. In a case where the number-average diameter is large, an occupied volume proportion of the fillers in the resin portion 19 becomes larger.

The fillers are included in the acoustic lens 20 and the resin portion 19. As the number-average diameter of the fillers becomes large, the ultrasonic waves are gradually scattered to be significantly attenuated. The filler included in the acoustic lens 20 is set as the first filler 39 and the filler included in the resin portion 19 is set as the second filler 40. A number-average diameter relationship of the filler for the second filler 40 is larger than that of the first filler 39. This may be regarded as the same meaning that occupied volume of the second filler 40 in the resin portion 19 is larger than occupied volume of the first filler 39 in the acoustic lens 20. When the occupied volume ratio of the filler is large, it is possible to improve diffusion efficiency of the ultrasonic waves and attenuate the ultrasonic waves. For this reason, it is possible to effectively attenuate the ultrasonic waves by using the resin portion 19 rather than the acoustic lens 20. With this, it is possible to suppress reflection of the ultrasonic waves on the acoustic lens end face 20a.

The ultrasonic probe 13 includes the first substrate 22 and the second substrate 23 at a side opposite to the emission direction 18 of the ultrasonic waves. The second substrate 23 plays a role of improving the stiffness of the first substrate 22. The ultrasonic waves emitted from the ultrasonic element 25 propagate also inside of the second substrate 23 and is reflected also on the second substrate end face 23a. The ultrasonic waves emitted from the ultrasonic element 25 is reflected also on the first substrate end face 22a of the first substrate 22 on which the ultrasonic element 25 is installed. The resin portion 19 is extended to the place opposing the first substrate 22 and the second substrate 23 so as to make it possible to effectively attenuate reflection of the ultrasonic waves on the second substrate end face 23a or the first substrate end face 22a of the first substrate 22 on which the ultrasonic element 25 is installed.

As described above, according to this embodiment, the following effects are obtained.

(1) According to this embodiment, the acoustic lens 20 has the acoustic lens end face 20a in the direction 28 intersecting the emission direction 18. The ultrasonic probe 13 includes the resin portion 19 which is opposed to the acoustic lens end face 20a and includes the filler and resin. The groove portion 24 formed between the ultrasonic device 17 and the probe casing 16 is filled with the resin portion 19. The filler is included in the resin portion 19 and the ultrasonic waves are irregularly reflected within the resin portion 19 by the filler. The ultrasonic waves are attenuated while advancing in the inside of the resin portion 19. By doing this, the resin portion 19 absorbs the ultrasonic waves. For that reason, the ultrasonic waves advancing toward an acoustic lens end face 20a are absorbed by the resin portion 19. Accordingly, reflection of the ultrasonic waves on the acoustic lens end face 20a is effectively attenuated. The resin portion 19 containing the filler is installed on the acoustic lens end face 20a and thus, reflection of the ultrasonic waves is suppressed. As a result, reflection of the ultrasonic waves on the acoustic lens end face 20a is suppressed and thus, accuracy of ultrasonic wave measurement can be improved.

(2) According to this embodiment, the filler is included in the acoustic lens 20 and the resin portion 19. As the number-average diameter of the filler is large, the ultrasonic waves are gradually scattered to be significantly attenuated. In a case where the filler included in the acoustic lens 20 is set as the first filler 39 and the filler included in the resin portion 19 is set as the second filler 40, a number-average diameter relationship of the filler for the second filler 40 is larger than that of the first filler 39. For this reason, it is possible to effectively attenuate the ultrasonic waves by using the resin portion 19 rather than the acoustic lens 20. With this, it is possible to suppress reflection of the ultrasonic waves on the acoustic lens end face 20a.

(3) According to this embodiment, silicone rubber, foamed silicone, and epoxy resin which are materials of the resin portion 19 have a nature of maintaining a state of being viscous body before curing and becoming solid after curing. For that reason, the filler can be easily mixed in the resin portion 19 before curing of the materials of the resin portion 19. Furthermore, moisture or foreign substances can be prevented from entering the inside of the ultrasonic probe 13 by the resin portion 19. Accordingly, the filler can be easily mixed in the resin portion 19 before curing and furthermore, moisture or foreign substances can be prevented from entering the inside of the ultrasonic probe 13 by the resin portion 19.

(4) According to this embodiment, the ultrasonic probe 13 includes the first substrate 22 and the second substrate 23 at a side opposite to the emission direction 18 of the ultrasonic waves. The second substrate 23 plays a role of improving the stiffness of the first substrate 22. The ultrasonic waves emitted from the ultrasonic element 25 propagate also inside of the second substrate 23 and are reflected also on the second substrate end face 23a. The ultrasonic waves emitted from the ultrasonic element 25 are reflected also on the first substrate end face 22a of the first substrate 22 on which the ultrasonic element 25 is installed. The resin portion 19 is extended to the place opposing the first substrate 22 and the second substrate 23 so as to make it possible to effectively attenuate reflection of the ultrasonic waves on the second substrate end face 23a or the first substrate end face 22a of the first substrate 22 on which the ultrasonic element 25 is installed.

### Embodiment 2

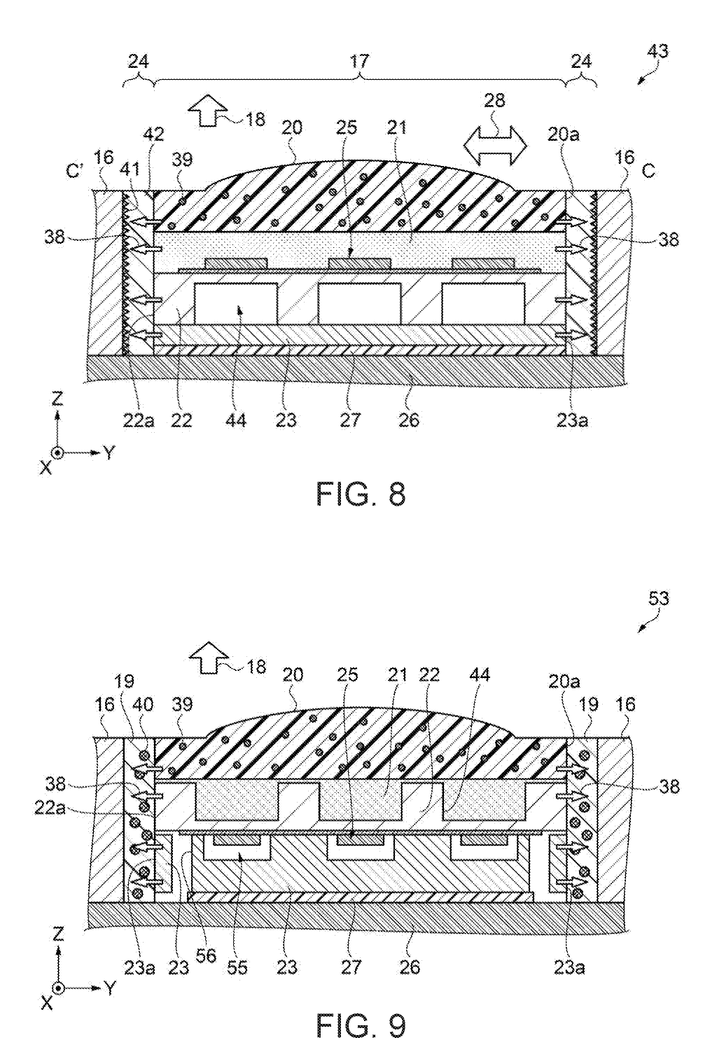

Next, one embodiment of the ultrasonic probe will be described using FIG. 7 and FIG. 8. The present embodiment is different from Embodiment 1 in that a recessed and projecting member 41 which attenuates the ultrasonic waves is installed. Also, description on the same matters as those of Embodiment 1 will be omitted.

FIG. 7 is a plan view schematically illustrating an ultrasonic probe according to Embodiment 2. The ultrasonic device 17 of an ultrasonic probe 43 is installed at the opening 16a of the probe casing 16. When the ultrasonic device 17 is installed at the opening 16a, the groove portion 24 is formed between the probe casing 16 and the ultrasonic device 17. The groove portion 24 is filled with the filling agent 42. The acoustic lens 20 installed in the ultrasonic device 17 is disposed to be exposed from the ultrasonic probe 43. In a case where an end face of the acoustic lens 20, which is positioned in the emission direction 18 of the ultrasonic waves emitted by the ultrasonic element 25 and has end face in the direction 28 intersecting the emission direction, is set as the acoustic lens end face 20a, the ultrasonic device 17 includes the recessed and projecting member 41 which is opposed to the acoustic lens end face 20a of the acoustic lens 20 and unevenness.

FIG. 8 is a cross-sectional view schematically illustrating a function of a recessed and projecting member according to Embodiment 2 and is a diagram when seen from a cross-section along line C-C′ of FIG. 7. The acoustic lens 20 has acoustic lens end face 20a in a direction 28 intersecting the emission direction. The recessed and projecting member 41 is installed at the opening 16a of the probe casing 16. The groove portion 24 formed between the probe casing 16 and the ultrasonic device 17 is filled with a filling agent 42. Although the filler is included in the resin portion 19 of Embodiment 1, the filler may not be included in the filling agent 42. The recessed and projecting member 41 irregularly reflects the ultrasonic waves. For that reason, the ultrasonic waves 38 advancing toward the acoustic lens end face 20a is irregularly reflected on the recessed and projecting member 41. Reflection on the acoustic lens end face 20a is effectively attenuated by the recessed and projecting member 41. The recessed and projecting member 41 is installed at the opening 16a of the probe casing 16 and thus, reflection of the ultrasonic waves is suppressed. As a result, reflection of the ultrasonic waves on the acoustic lens end face 20a is suppressed and thus, accuracy of ultrasonic wave measurement can be improved.

A cross-section of unevenness of the recessed and projecting member 41 is preferably a triangle in at least one of a direction orthogonal to or parallel with the acoustic lens end face. A height of the recessed and projecting member 41 may be available as long as it is the integer multiple of a wavelength λ of the ultrasonic waves. A shape of which a cross-section in at least one of the orthogonal direction and the parallel direction with respect to the acoustic lens end face 20a becomes a triangle is, for example, a triangular pyramid or a triangular prism.

The ultrasonic probe 43 includes the acoustic lens 20 and the recessed and projecting member 41. When a cross-section in at least one of the orthogonal direction and the parallel direction with respect to the acoustic lens end face 20a is a triangle, the recessed and projecting member 41 can irregularly reflect the ultrasonic waves effectively. The ultrasonic waves 38 advancing toward the acoustic lens end face 20a is irregularly reflected on the recessed and projecting member 41 and thus, reflection of the ultrasonic waves toward the acoustic lens end face 20a is effectively attenuated. The recessed and projecting member 41 is installed at the opening 16a of the probe casing 16 and thus, reflection of the ultrasonic waves on the acoustic lens end face 20a is suppressed.

The first substrate 22 on which the ultrasonic element 25 is installed and the second substrate 23 for improving the stiffness of the first substrate 22 are installed in this order at a side opposite to the emission direction 18 of the ultrasonic waves of the acoustic lens 20. The first substrate 22 and the second substrate 23 have end faces in the direction 28 intersecting the emission direction, the end face of the first substrate 22 is set as the first substrate end face 22a, and the end face of the second substrate 23 is set as the second substrate end face 23a. The recessed and projecting member 41 is preferably opposed to the first substrate end face 22a and the second substrate end face 23a.

The ultrasonic probe 43 includes the first substrate 22 and the second substrate 23 at a side opposite to the emission direction 18 of the ultrasonic waves. The ultrasonic element 25 is installed on the first substrate 22. The second substrate 23 plays a role of improving the stiffness of the first substrate 22. The ultrasonic waves emitted from the ultrasonic element 25 propagate also inside of the second substrate 23 and are reflected also on the second substrate end face 23a. The ultrasonic waves emitted from the ultrasonic element 25 are reflected also on the first substrate end face 22a of the first substrate 22 on which the ultrasonic element 25 is installed. The recessed and projecting member 41 is extended to the place opposing the first substrate 22 and the second substrate 23. Accordingly, it is possible to effectively attenuate reflection of the ultrasonic waves on the second substrate end face 23a or the first substrate end face 22a by the recessed and projecting member 41.

In the ultrasonic probe 43, the probe casing 16 is preferably installed to be opposed to the acoustic lens end face 20a and the recessed and projecting member 41 is preferably integrated with the probe casing 16. The probe casing 16 may be composed of ABS resin, acrylic resin, or a resin material. The probe casing 16 may also be composed of a composite material of silicone rubber and the resin material.

The probe casing 16 is preferably installed to be opposed to the acoustic lens end face 20a in the ultrasonic probe 43. The probe casing 16 includes the recessed and projecting member 41 at places thereof opposing the acoustic lens end face 20a. The recessed and projecting member 41 irregularly reflects the ultrasonic waves. For that reason, the ultrasonic waves 38 advancing toward the acoustic lens end face 20a are irregularly reflected on the recessed and projecting member 41 and thus, reflection of the ultrasonic waves on the acoustic lens end face 20a is effectively attenuated. The recessed and projecting member 41 is installed at the opening 16a of the probe casing 16 and thus, reflection of the ultrasonic waves can be suppressed. Since the recessed and projecting member 41 is integrated with the end face of the probe casing 16, the number of components can be reduced compared to a case where the recessed and projecting member 41 and the end face of the probe casing 16 are formed at different portions and accordingly, it is possible to manufacture the ultrasonic probe 43 with high productivity.

As described above, the following effects are obtained according to this embodiment.

(1) According to this embodiment, the ultrasonic probe 43 includes the acoustic lens 20 and the recessed and projecting member 41. The acoustic lens 20 has the acoustic lens end face 20a in the direction 28 intersecting the emission direction. The recessed and projecting member 41 is disposed at the opening 16a of the probe casing 16. The groove portion 24 formed between the ultrasonic device 17 and the probe casing 16 is filled with the filling agent 42. Although the filler is included in the resin portion 19 of Embodiment 1, the filler may not be included in the filling agent 42.

The recessed and projecting member 41 irregularly reflects the ultrasonic waves. For that reason, the ultrasonic waves 38 advancing toward the acoustic lens end face 20a are irregularly reflected on the recessed and projecting member 41. With this, reflection on the acoustic lens end face 20a is effectively attenuated. The recessed and projecting member 41 is installed at the opening 16a of the probe casing 16 and thus, reflection of the ultrasonic waves is suppressed. As a result, reflection of the ultrasonic waves on the acoustic lens end face 20a is suppressed and thus, accuracy of ultrasonic wave measurement can be improved.

(2) According to this embodiment, the cross-section of unevenness of the recessed and projecting member 41 is preferably a triangle in at least one of a direction orthogonal to or parallel with the acoustic lens end face 20a. When the cross-section of unevenness of the recessed and projecting member 41 in at least one of a direction orthogonal to or parallel with the acoustic lens end face 20a is a triangle, it is possible to irregularly reflect the ultrasonic waves effectively. Accordingly, the ultrasonic waves advancing toward the acoustic lens end face 20a are irregularly reflected on the recessed and projecting member 41 and thus, reflection of the ultrasonic waves on the acoustic lens end face 20a is effectively attenuated.

(3) According to this embodiment, the ultrasonic probe 43 includes the first substrate 22 and the second substrate 23 at a side opposite to the emission direction 18 of the ultrasonic waves. The ultrasonic element 25 is installed on the first substrate 22. The second substrate 23 plays a role of improving the stiffness of the first substrate 22. The ultrasonic waves emitted from the ultrasonic element 25 propagate also inside of the second substrate 23 and are reflected also on the second substrate end face 23a. The ultrasonic waves emitted from the ultrasonic element 25 are reflected also on the first substrate end face 22a of the first substrate 22 on which the ultrasonic element 25 is installed. The recessed and projecting member 41 is extended to the place opposing the first substrate 22 and the second substrate 23 so as to make it possible to effectively attenuate reflection of the ultrasonic waves on the second substrate end face 23a or the first substrate end face 22a.

(4) According to this embodiment, the probe casing 16 is installed to be opposed to the acoustic lens end face 20a, in the ultrasonic probe 43. The ultrasonic probe 43 includes the recessed and projecting member 41 which is opposed to the acoustic lens end face 20a. The recessed and projecting member 41 irregularly reflects the ultrasonic waves. For that reason, the ultrasonic waves 38 advancing toward the acoustic lens end face 20a are irregularly reflected on the recessed and projecting member 41 and thus, reflection of the ultrasonic waves on the acoustic lens end face 20a is effectively attenuated. The recessed and projecting member 41 is installed at the opening 16a of the probe casing 16 and thus, reflection of the ultrasonic waves can be suppressed. Since the recessed and projecting member 41 is integrated with the end face of the probe casing 16, the number of components can be reduced compared to a case where the recessed and projecting member 41 and the end face of the probe casing 16 are formed at different portions and accordingly, it is possible to manufacture the ultrasonic probe 43 with high productivity.

The invention is not limited to the embodiments described above and various alterations or improvements can be made to the embodiments described above. In the following, modification examples will be described in detail.

### MODIFICATION EXAMPLE 1

In Embodiment 1 described above, matters that at least any one of silicone rubber, foamed silicone, and epoxy resin is included, as the material of the resin portion 19, are described, but are not limited to the configuration thereof. In the following, the ultrasonic probe 13 according to Modification Example 1 will be described. The same constitutional elements as those of Embodiment 1 are assigned the same reference numerals and redundant description will be omitted.

Silicone rubber, foamed silicone, and epoxy resin are curable resin having a nature of maintaining a state of being viscous body before curing and becoming solid after curing and thus, the filler can be easily mixed. Other materials which are curing resin may be used. By doing this, the filler can be easily mixed before curing. Furthermore, moisture or foreign substances can be prevented from entering the inside of the ultrasonic probe 13. Accordingly, the filler can be easily mixed in the resin portion 19 before curing and furthermore, moisture or foreign substances can be prevented from entering the inside of the ultrasonic probe 13.

### MODIFICATION EXAMPLE 2

In Embodiment 1 described above, matters that at least any one of powders of a group of zinc oxide powder, zirconium oxide powder, alumina powder, silica powder, titanium oxide powder, silicon carbide powder, aluminum nitride powder, carbon powder, calcium carbonate powder, and boron nitride powder is included in the filler, which is included in the resin portion 19 are described, but are not limited to the configuration thereof. In the following, the ultrasonic probe 13 according to Modification Example 2 will be described.

The size of the particle diameter of various fillers included in the resin portion 19 is the same as or equal to or less than the wavelength of the ultrasonic waves and thus, various fillers have a nature of diffusing the ultrasonic waves. In addition to the fillers described above, as long as the size of the particle diameter of the fillers is the same as or equal to or less than the wavelength of the ultrasonic waves and the fillers have a nature of diffusing the ultrasonic wave, these fillers may be used. These fillers can scatter and attenuate the ultrasonic waves. For that reason, the resin portion 19 can attenuate the ultrasonic waves.

### MODIFICATION EXAMPLE 3

FIG. 9 illustrates a cross-sectional view schematically illustrating an ultrasonic probe according to Modification Example 3. In Embodiment 1 described above, a structure of the ultrasonic probe 13 is described, but is not limited to the configuration thereof. In the following, an ultrasonic probe 53 according to Modification Example 3 will be described.

In the ultrasonic probe 53, the acoustic lens 20, the acoustic matching layer 21, the first substrate 22, and the second substrate 23 are installed to be superimposed on one another in this order. A recessed portion 55 is installed on the second substrate 23 and the ultrasonic element 25 is positioned inside the recessed portion 55. The first substrate 22 includes a recessed portion 44. The recessed portion 44 side of the first substrate 22 corresponds to the emission direction 18. Accordingly, the ultrasonic waves advance in order of the recessed portion 44, the acoustic matching layer 21, and the acoustic lens 20 to be emitted.

The acoustic matching layer 21 disposed between the first substrate 22 and the acoustic lens 20 is filled into the inside of the recessed portion 44. The ultrasonic waves generated by the ultrasonic element 25 may be emitted through the acoustic matching layer 21 filled into the recessed portion 44. By doing this, it is possible to protect the ultrasonic element 25 from moisture or foreign substances entering from outside.

Also, the ultrasonic probe 53 is configured in such away that the resin portion 19 is installed at places opposing the second substrate end face 23a, the first substrate end face 22a, and the acoustic lens end face 20a. Accordingly, the unnecessary ultrasonic waves advancing toward the second substrate end face 23a, the first substrate end face 22a, and the acoustic lens end face 20a can be attenuated by the resin portion 19.

### MODIFICATION EXAMPLE 4

In Embodiment 2 described above, matters that the cross-section of unevenness of the recessed and projecting member 41 is a triangle in at least one of a direction orthogonal to or parallel with the acoustic lens end face 20a are described, but are not limited to the configuration thereof. In the following, the ultrasonic probe according to Modification Example 4 will be described.

The cross-section of unevenness of the recessed and projecting member 41 in at least one of a direction orthogonal to or parallel with the acoustic lens end face 20a is not limited to a triangle, but may include a circle, a quadrangle, or a polygon. By doing this, the ultrasonic waves 38 advancing toward the acoustic lens end face 20a is irregularly reflected on the recessed and projecting member 41 and thus, reflection of the ultrasonic waves toward the acoustic lens end face 20a is effectively attenuated. The recessed and projecting member 41 is installed at the opening 16a of the probe casing 16 and thus, reflection of the ultrasonic waves is suppressed.

### MODIFICATION EXAMPLE 5

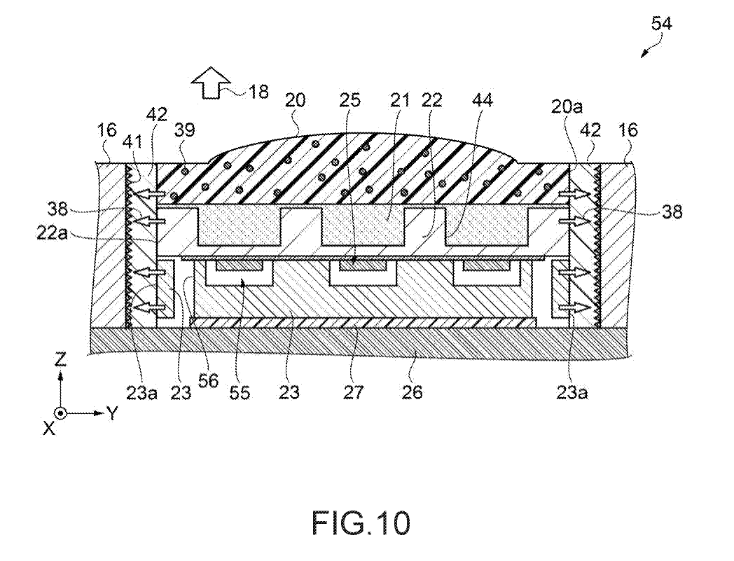

FIG. 10 illustrates a cross-sectional view schematically illustrating an ultrasonic probe according to Modification Example 5. In Embodiment 2 described above, the structure of the ultrasonic probe 43 is described, but is not limited to the configuration thereof.

In the following, the ultrasonic probe 54 according to Modification Example 5 will be described.

In the ultrasonic probe 54, the acoustic lens 20, the acoustic matching layer 21, and the first substrate 22 are installed to be superimposed on one another in this order. Similar to Modification Example 3, the first substrate 22 includes the recessed portion 44. The recessed portion 44 side of the first substrate 22 corresponds to the emission direction 18. Accordingly, the ultrasonic waves advance in order of the recessed portion 44, the acoustic matching layer 21, and the acoustic lens 20 to be emitted.

The acoustic matching layer 21 disposed between the first substrate 22 and the acoustic lens 20 is filled into the inside of the recessed portion 44. The ultrasonic waves generated by the ultrasonic element 25 may be emitted through the acoustic matching layer 21 filled into the recessed portion 44. By doing this, it is possible to protect the ultrasonic element 25 from moisture or foreign substances entering from outside.

Also, the ultrasonic probe 54 is configured in such a way that the recessed and projecting member 41 is installed at places opposing the second substrate end face 23a, the first substrate end face 22a, and the acoustic lens end face 20a. Accordingly, the unnecessary ultrasonic waves advancing toward the second substrate end face 23a, the first substrate end face 22a, and the acoustic lens end face 20a can be attenuated by the recessed and projecting member 41.

The cross-section of unevenness of the recessed and projecting member 41 in at least one of a direction orthogonal to or parallel with the acoustic lens end face 20a is not limited to a triangle, but may include a circle, a quadrangle, or a polygon. By doing this, the ultrasonic waves 38 advancing toward the acoustic lens end face 20a are irregularly reflected on the recessed and projecting member 41 and thus, reflection of the ultrasonic waves toward the acoustic lens 20 from the recessed and projecting member 41 is effectively attenuated. The recessed and projecting member 41 is installed at the opening 16a of the probe casing 16 and thus, reflection of the ultrasonic waves is suppressed. Through holes 56 penetrating through in the Z-direction are installed on the second substrate 23 of FIG. 9 and FIG. 10. The through holes 56 are holes used for installing wirings. When a place where wirings are to be installed can be provided at a portion other than the second substrate 23, the through holes 56 may not necessarily be installed on the second substrate 23.

The entire disclosure of Japanese Patent Application No. 2016-241960 filed Dec. 14, 2016 is expressly incorporated by reference herein.

What is claimed is:

1. An ultrasonic probe comprising:
an acoustic lens which is positioned in an emission direction of ultrasonic waves emitted by an ultrasonic element and has end face in a direction intersecting the emission direction; and
a resin portion which is opposed to acoustic lens end face which is the end face of the acoustic lens and includes a filler and resin.

2. The ultrasonic probe according to claim 1,
wherein the acoustic lens includes a filler, and in a case where the filler included in the acoustic lens is set as a first filler and the filler included in the resin portion is set as a second filler, the number-average diameter of the second filler is larger than the number-average diameter of the first filler.

3. The ultrasonic probe according to claim 1,
wherein the material of the resin portion includes at least any one of silicone rubber, foamed silicone, and epoxy resin and the filler included in the resin portion includes at least any one of powders of the group consisting of zinc oxide powder, zirconium oxide powder, alumina powder, silica powder, titanium oxide powder, silicon carbide powder, aluminum nitride powder, carbon powder, calcium carbonate powder, and boron nitride powder.

4. The ultrasonic probe according to claim 1,
wherein a first substrate on which the ultrasonic element is installed and a second substrate for improving the stiffness of the first substrate are installed in this order at a side opposite to the emission direction of the ultrasonic waves of the acoustic lens,
the first substrate and the second substrate have end faces in a direction intersecting the emission direction, and
in a case where the end face of the first substrate is set as a first substrate end face and the end face of the second substrate is set as a second substrate end face, the resin portion is opposed to the first substrate end face and the second substrate end face.

5. An ultrasonic probe comprising:
an acoustic lens which is positioned in an emission direction of the ultrasonic waves emitted by an ultrasonic element and has an acoustic lens end face in the direction intersecting the emission direction; and
a recessed and projecting member which is opposed to the acoustic lens end face and has unevenness.

6. The ultrasonic probe according to claim 5,
wherein a cross-section of unevenness of the recessed and projecting member is a triangle in at least one of a direction orthogonal to or parallel with the acoustic lens end face.

7. The ultrasonic probe according to claim 5,
wherein a first substrate on which the ultrasonic element is installed and a second substrate for improving the stiffness of the first substrate are installed in this order at a side opposite to the emission direction of the ultrasonic waves of the acoustic lens,
the first substrate and the second substrate have end faces in a direction intersecting the emission direction, and
in a case where the end face of the first substrate is set as the first substrate end face and the end face of the second substrate is set as the second substrate end face, the recessed and projecting member is opposed to the first substrate end face and the second substrate end face.

8. The ultrasonic probe according to claim 5,
wherein a probe casing is installed to be opposed to the acoustic lens end face, and
the recessed and projecting member is integrated with the probe casing.
